(19) United States
(12) Patent Application Publication
(10) Pub. No.: US 2013/0102773 A1
(43) Pub. Date: Apr. 25, 2013

(54) METHOD OF PREPARING SUCRALOSE-6-ESTER BY CATALYSIS AND CHLORINATION OF PHASE TRANSFER CATALYST

(71) Applicant: Zhengyou WANG, HUBEI PROVINCE (CN)

(72) Inventor: Zhengyou WANG, HUBEI PROVINCE (CN)

(73) Assignee: HUBEI YITAI PHARMACEUTICAL CO.,LTD., HUBEI PROVINCE (CN)

(21) Appl. No.: 13/558,357

(22) Filed: Jul. 26, 2012

(30) Foreign Application Priority Data
Oct. 19, 2011 (CN) 201110318595.1

(51) Int. Cl. C07H 1/00

(52) U.S. Cl. 536/115

(57) ABSTRACT

A method of preparing sucralose-6-ester by catalysis and chlorination of phase transfer catalyst comprises the following steps: add phase transfer catalyst into a prepared Vilsmeier reagent and then drip in DMF solution of sucrose-6-ester at 5-10° C. to obtain a feed solution and after that maintain its temperature of the feed solution for 1-1.5 hours; next, increase temperature of the feed solution to 45-55° C. and maintain the temperature for 1-1.5 hours; next, increase the temperature to 75-85° C. and maintain the temperature for 1-1.5 hours; next, increase the temperature to 108-109° C., maintain the temperature for 2.5-3 hours and then remove a chlorine-containing solvent from the feed solution by concentration in reduced pressure for evaporation. After that, cool down the feed solution to room temperature and then pump in ammonia into the feed solution for neutralization.

### BACKGROUND OF THE INVENTION

The present invention relates to the technical field of preparing sucralose, and more specifically relates to a kind of method of preparing sucralose-6-ester by catalysis and chlorination of phase transfer catalyst.

In the field of preparing sucralose, monoesterification is very common in industrial use. Its process includes: transform sucrose into sucrose-6-ester and then chlorinate the sucrose-6-ester to obtain sucralose-6-ester, which is then subject to alcoholysis such that sucralose is prepared. Among the said process, chlorination of the sucrose-6-ester is a crucial step and chlorination method is therefore very important. Vilsmeier reagent is commonly used in the chlorination of sucrose-6-ester. Vilsmeier reagent is prepared by reaction between dimethylformamide (DMF) and thionyl chloride or bis (trichloromethyl) carbonate. During reaction between the Vilsmeier reagent and the sucrose-6-ester, the Vilsmeier reagent and sucrose-6-ester solution could not satisfactorily dissolve each other under 50° C. and it is relatively difficult to attain homogeneous reaction, resulting in unstable reaction process. Therefore, temperature is required to be under 5° C. when DMF solution of the sucrose-6-ester is added, and also, a longer period of time (10 to 13 hours) is required for feed solution to be heated stage by stage from 5° C. to 110° C. gradually so as to prevent hazards due to unstable reaction. Also, effective transformation into sucralose-6-ester could only be attained when final temperature is above 110° C. and a temperature from 110° C. to 113° C. should also be maintained for reaction for 3 hours. Because of long reaction in such a relatively high temperature, relatively more by-products are produced during the process, resulting in serious carbonation of material in the feed solution in which deep black colloidal substances emerge. Accordingly, product yield decreases and post-treatment of the feed solution is difficult.

Moreover, during post-treatment after chlorination is completed, a 30% liquid caustic soda neutralizing feed solution is often used, and such kind of treatment has its disadvantage because the liquid caustic soda brings in much water into the feed solution leading possibly to side effect of hydrolysis. Besides, as DMF and water are mutually dissolved, not only is DMF difficult to be recycled, chlorinated products are partially carried away by water phase of skimming during extraction.

### BRIEF SUMMARY OF THE INVENTION

In view of the aforementioned disadvantages now present in the prior arts, the present invention provides a kind of method of preparing sucralose-6-ester by catalysis and chlorination of phase transfer catalyst.

A method of preparing sucralose-6-ester by catalysis and chlorination of phase transfer catalyst comprises the following steps: Add phase transfer catalyst with sucrose-6-ester weighing 2-3% into a prepared Vilsmeier reagent and then drip in DMF solution of sucrose-6-ester with concentration of 15-25% in 5-10° C. to obtain a feed solution, and after that maintain temperature of the feed solution for 1-1.5 hours; next, increase the temperature to 45-55° C. and maintain the temperature for 1-1.5 hours; next, increase the temperature to 75-85° C. and maintain the temperature for 1-1.5 hours; next, increase the temperature to 108-109° C. , maintain the temperature for 2.5-3 hours and then remove a chlorine-containing solvent (dichloroethane or trichloroethane) from the feed solution by concentration in reduced pressure for evaporation.

Cool down the feed solution to room temperature, pump in ammonia to the feed solution for neutralization, remove salt from the feed solution by centrifugation, remove the DMF solution from the feed solution for recycling by concentration in reduced pressure under high vacuum below 60° C., dissolve residues of the feed solution by using ethyl acetate, wash it twice by purified water, add in active carbon to decolorize and after that remove ethyl acetate solvent under vacuum, add water to dissolve residues and then cool down for crystallization; and suralose-6-ester is finally obtained.

After the ammonia is pumped in, pH value of the feed solution is 9.0-9.5 and then adjusted to 6.5-7.5. The feed solution is then stirred for 1 hour.

The Vilsmeier chlorination reagent is prepared by first dissolving a chlorination reagent in a chlorine-containing solvent, namely dichloroethane or trichloroethane, which weighs two to three times more than the chlorination reagent, and then dripping resultant solvent into a DMF solvent.

The chlorination reagent is one of the following: thionyl chloride, bis (trichloromethyl) carbonate or phosphorus oxychloride.

The phase transfer catalyst is one of the following: tetrabutyl ammonium bromide, tetrabutyl ammonium chloride, benzyl triethyl ammonium chloride, benzyl triethyl ammonium bromide, trioctylmethyl ammonium chloride, dodecyltrimethyl ammonium chloride, trimethyl-tetradecyl ammonium chloride.

As compared with prior arts, the present invention is advantageous in that:

Since phase transfer catalyst is added into feed solution during chlorination, reaction rate between sucrose-6-ester and chlorination agent is increased, DMF solution of sucrose-6-ester could be dripped in at a slightly higher temperature of 5-10° C. during production, and low temperature reaction is more stable and also safer. Besides, after esterified solution is dripped in, only 6-8 hours are required to reach the highest temperature at 108-109° C. for reaction. The final temperature of reaction is thus decreased by 5° C. and the temperature maintaining time is shortened by 0.5 hour, and these advantages could save energy consumption. Further, due to reduced emergence of by-products, namely black colloidal substances, post-treatment is also relatively simpler.

During post-treatment of chlorinated feed solution, ammonia is first pumped in for neutralization and products are eventually dissolved by using ethyl acetate after DMF solvent is evaporated and recycled. By using such method, no water is dragged in so that dissolution and loss of sucralose-6-ester are reduced. Further, recycling costs of DMF solvent are reduced, steam energy consumption would also be reduced by 20-30% when DMF is distilled and recycled. Accordingly, total production cost is reduced.

By using phase transfer catalyst such as tetrabutyl ammonium bromide, tetrabutyl ammonium chloride, benzyl triethyl ammonium chloride, benzyl triethyl ammonium bromide, trioctylmethyl ammonium chloride, dodecyltrimethyl ammonium chloride, trimethyl-tetradecyl ammonium chloride and so forth, substances could be facilitated to be dissolved more satisfactorily and there could be stable and efficient chlorination reaction process.

### DETAILED DESCRIPTION OF THE INVENTION

The present invention is further described below via the following embodiments but the present invention is not limited to the described embodiments.

### Embodiment 1

A method of preparing sucralose-6-ester by catalysis and chlorination of phase transfer catalyst comprises the following steps: Add phase transfer catalyst with sucrose-6-ester weighing 2% into a prepared Vilsmeier reagent and then drip in DMF solution of sucrose-6-ester with weight percent concentration of 15% in 5° C. to obtain a feed solution, and after that maintain temperature of the feed solution for 1 hour; next, increase the temperature to 45° C. and maintain the temperature for 1 hour; next, increase the temperature to 75° C. and maintain the temperature for 1 hour; next, increase the temperature to 108° C. , maintain the temperature for 2.5 hours and then remove a chlorine-containing solvent (dichloroethane or trichloroethane) from the feed solution by concentration in reduced pressure for evaporation; next, cool down the feed solution to room temperature, pump in ammonia to the feed solution for neutralization, filter the feed solution to remove salt, remove the DMF solution from the feed solution for recycling by concentration in reduced pressure under high vacuum below 60° C., dissolve residues of the feed solution by using ethyl acetate, wash it twice by purified water, add in active carbon to decolorize and after that remove ethyl acetate solvent under vacuum, add water to dissolve residues and then cool down for crystallization; and suralose-6-ester is finally obtained.

### Embodiment 2

A method of preparing sucralose-6-ester by catalysis and chlorination of phase transfer catalyst comprises the following steps: Add phase transfer catalyst with sucrose-6-ester weighing 3% into a prepared Vilsmeier reagent and then drip in DMF solution of sucrose-6-ester with weight percent concentration of 25% in 10° C. to obtain a feed solution and after that maintain temperature of the feed solution for 1 hour; next, increase the temperature to 55° C. and maintain the temperature for 1.5 hours; next, increase the temperature to 85° C. and maintain the temperature for 1.5 hours; next, increase the temperature to 109° C., maintain the temperature for 3 hours and then remove a chlorine-containing solvent (dichloroethane or trichloroethane) from the feed solution by concentration in reduced pressure for evaporation; next, cool down the feed solution to room temperature, pump in ammonia to the feed solution for neutralization, filter the feed solution to remove salt, remove the DMF solution from the feed solution for recycling by concentration in reduced pressure under high vacuum below 60° C., dissolve residues of the feed solution by using ethyl acetate, wash it twice by purified water, add in active carbon to decolorize and after that remove ethyl acetate solvent under vacuum, add water to dissolve residues and then cool down for crystallization; and suralose-6-ester is finally obtained.

### Embodiment 3

A method of preparing sucralose-6-ester by catalysis and chlorination of phase transfer catalyst comprises the following steps: Add phase transfer catalyst with sucrose-6-ester weighing 2.5% into a prepared Vilsmeier reagent and then drip in DMF solution of sucrose-6-ester with weight percent concentration of 20% in 8° C. to obtain a feed solution and after that maintain temperature of the feed solution for 1.2 hours; next, increase the temperature to 50° C. and maintain the temperature for 1.2 hours; next, increase the temperature to 80° C. and maintain the temperature for 1.2 hours; next, increase the temperature to 108° C., maintain the temperature for 2.8 hours and then remove a chlorine-containing solvent (dichloroethane or trichloroethane) from the feed solution by concentration in reduced pressure for evaporation; next, cool down the feed solution to room temperature, pump in ammonia to the feed solution until the feed solution has a pH value of 9.0-9.5; the feed solution is then neutralized and the pH value is adjusted to be 6.5-7.5; the feed solution is then stirred for 1 hour; next, filter the feed solution to remove salt, remove the DMF solution from the feed solution for recycling by concentration in reduced pressure under high vacuum below 60° C., dissolve residues of the feed solution by using ethyl acetate, wash it twice by purified water, add in active carbon to decolorize and after that remove ethyl acetate solvent under vacuum, add water to dissolve residues and then cool down for crystallization; and suralose-6-ester is finally obtained.

The Vilsmeier reagent is prepared by first dissolving a chlorination reagent in a chlorine-containing solvent, namely dichloroethane or trichloroethane, which weighs two to three times more than the chlorination reagent, and then dripping resultant solvent into a DMF solvent.

The aforementioned phase transfer catalyst could be one of the following: tetrabutyl ammonium bromide, tetrabutyl ammonium chloride, benzyl triethyl ammonium chloride, benzyl triethyl ammonium bromide, trioctylmethyl ammonium chloride, dodecyltrimethyl ammonium chloride, trimethyl-tetradecyl ammonium chloride.

As mentioned before, the Vilsmeier reagent is prepared by first dissolving a chlorination reagent in a chlorine-containing solvent chlorine, namely, dichloroethane or trichloroethane, and then dripping resultant solvent into a DMF solvent. The said chlorination reagent is one of the following: thionyl chloride, bis (trichloromethyl) carbonate or phosphorus oxychloride. Moreover, after pumping in ammonia, pH value of the feed solution is 9.0-9.5 and then adjusted to 6.5-7.5, and the feed solution is then stirred for 1 hour.

### Embodiment 4

Dissolve 40 g of sucrose-6-ester in 200 ml of DMF (N,N-dimethylformamide) and reserve resultant solution for subsequent use.

Under temperature of 0-5° C., drip bis (trichloromethyl) carbonate solution (125 g of bis (trichloromethyl) carbonate dissolved in 200 ml of dichloroethane under room temperature) into 300 ml of DMF for 1 hour and stir for 2 hours under room temperature such that Vilsmeier reagent is prepared; next, add in 0.4 g of tetrabutyl ammonium chloride, control such substance's temperature below 10° C., drip in the reserved sucrose-6-ester solution for an hour to complete the dripping procedure to obtain a feed solution and then maintain temperature of the feed solution for an hour; next; increase the temperature to 45-55° C. and maintain the temperature for 1 hour; next; increase the temperature to 75-85° C. and maintain the temperature for 1 hour; next, increase the temperature to 108-109° C., stir in maintained temperature for 2.5 hours and after that remove solvent dichloroethane from the feed solution by concentration in reduced pressure for evaporation; next, cool down the feed solution to room temperature, pump in ammonia to the feed solution until the feed solution has a pH value of 9.0-9.5; the feed solution is then neutralized and the pH value of the feed solution is adjusted to 6.5-7.5; the feed solution is then stirred for an hour; next, filter the feed solution to remove salt while filter cake is washed by a small amount of DMF.

Filtrate is then maintained at a temperature below 60° C. and then concentrated in reduced pressure till dried to recycle DMF solvent; next; dissolve residues 2 to 3 times by using ethyl acetate, combine all acetate layers and then wash the acetate layers twice by using purified water to remove water-soluble impurities; next, add active carbon to ethyl acetate phase for decolorization, stir for an hour. leach, wash, combine with acetate solution and then concentrate in vacuum till dried. After that, add 125 ml of water into residues, increase temperature to stir and dissolve, and a small amount of ethyl ether is then added in when temperature cools down below 40° C. By stirring, crystallization and filtration, raw sucralose-6-ester is obtained, ethyl acetate is re-crystallized and 25 g of refined sucralose-6-ester is obtained after drying (yield: 62.5%).

### Embodiment 5

Dissolve 40 g of sucrose-6-ester in 200 ml of DMF and reserve resultant solution for subsequent use.

Under temperature of 0-5° C., drip thionyl chloride solution (165 g of thionyl chloride dissolved in 200 ml of dichloroethane under room temperature) into 280 ml of DMF for 1 hour, stir and react for 2 hours such that Vilsmeier reagent is prepared; next, add in 0.4 g of tetrabutyl ammonium bromide, control such substance's temperature below 10° C., drip in the reserved sucrose-6-ester solution for an hour to complete the dripping procedure to obtain a feed solution and then maintain temperature of the feed solution for an hour; next; increase the temperature to 45-55° C. and maintain the temperature for 1 hour; next; increase the temperature to 75-85° C. and maintain the temperature for 1 hour; next, increase the temperature to 108-109° C., stir in maintained temperature for 2.5 hours and after that remove solvent dichloroethane from the feed solution by concentration in reduced pressure for evaporation; next, cool down the feed solution to room temperature, pump in ammonia to the feed solution until the feed solution has a pH value of 9.0-9.5; the feed solution is then neutralized and the pH value is adjusted to 6.5-7.5; the feed solution is then stirred for an hour; next, filter the feed solution to remove salt while filter cake is washed by a small amount of DMF.

Filtrate is then maintained at a temperature below 60° C. and then concentrated in reduced pressure till dried to recycle DMF solvent; next; dissolve residues 2 to 3 times by using ethyl acetate, combine all acetate layers and then wash the acetate layers twice by using purified water to remove water-soluble impurities; next, add active carbon to ethyl acetate phase for decolorization, stir for an hour, leach, wash, combine with acetate solution and then concentrate in vacuum till dried. After that, add 125 ml of water into residues, increase temperature to 65° C. to stir and dissolve, and a small amount of ethyl ether is then added in when temperature cools down below 40° C. By stirring, crystallization and filtration, raw sucralose-6-ester is obtained, ethyl acetate is re-crystallized and 21 g of refined sucralose-6-ester is obtained after drying (yield: 52.5%).

### Embodiment 6

Dissolve 40 g of sucrose-6-ester in 200 ml of DMF and reserve resultant solution for subsequent use.

Under temperature of 0-5° C., drip bis (trichloromethyl) carbonate solution (125 g of bis (trichloromethyl) carbonate dissolved in 200 ml of trichloroethane under room temperature) into 300 ml of DMF for 1 hour and stir for 2 hours under room temperature such that Vilsmeier reagent is prepared; next, add in 0.5 g of benzyl triethyl ammonium chloride, control such substance's temperature below 10° C., drip in the reserved sucrose-6-ester solution for an hour to complete the dripping procedure to obtain a feed solution and then maintain temperature of the feed solution for an hour; next; increase the temperature to 45-55° C. and maintain the temperature for 1 hour; next; increase the temperature to 75-85° C. and maintain the temperature for 1 hour; next, increase the temperature to 108-109° C. and maintain the temperature for 2.5 hours and after that remove solvent trichloroethane from the feed solution by concentration in reduced pressure for evaporation; next, cool down the feed solution to room temperature, pump in ammonia to the feed solution until the feed solution has a pH value of 9.0-9.5; the feed solution is then neutralized and the pH value is adjusted to be 6.5-7.5; the feed solution is then stirred for an hour; next, filter the feed solution to remove salt while filter cake is washed by a small amount of DMF.

Filtrate is then maintained at a temperature below 60° C. and then concentrated in reduced pressure till dried to recycle DMF solvent; next; dissolve residues 2 to 3 times by using ethyl acetate, combine all acetate layers and then wash the acetate layers twice by using purified water to remove water-soluble impurities; next, add active carbon to ethyl acetate phase for decolorization, stir for an hour, leach, wash, combine with acetate solution and then concentrate in vacuum till dried. After that, add 125 ml of water into residues, increase temperature to 65° C. to stir and dissolve, and a small amount of ethyl ether is then added in when temperature cools down below 40° C. By stirring, crystallization and filtration, raw sucralose-6-ester is obtained, ethyl acetate is re-crystallized and 24.1 g of refined sucralose-6-ester is obtained after drying (yield: 60%).

### Embodiment 7

Dissolve 40 g of sucrose-6-ester in 200 ml of DMF and reserve resultant solution for subsequent use.

Under temperature of 0-5° C., drip thionyl chloride solution (165 g of thionyl chloride dissolved in 200 ml of dichloroethane under room temperature) into 280 ml of DMF for 1 hour, stir and react for 2 hours such that Vilsmeier reagent is prepared; next, add in 0.5 g of benzyl triethyl ammonium chloride, control such substance's temperature below 10° C., drip in the reserved sucrose-6-ester solution for an hour to complete the dripping procedure to obtain a feed solution and then maintain temperature of the feed solution for an hour; next; increase the temperature to 45-55° C. and maintain the temperature for 1 hour; next; increase the temperature to 75-85° C. and maintain the temperature for 1 hour; next, increase the temperature to 108-109° C. and maintain the temperature for 2.5 hours and after that remove solvent dichloroethane from the feed solution by concentration in reduced pressure for evaporation; next, cool down the feed solution to room temperature, pump in ammonia until the feed solution has a pH value of 9.0-9.5; the feed solution is then neutralized and the pH value is adjusted to 6.5-7.5; the feed solution is then stirred for an hour; next, filter the feed solution to remove salt while filter cake is washed by a small amount of DMF.

Filtrate is then maintained at a temperature below 60° C. and then concentrated in reduced pressure till dried to recycle DMF solvent; next; dissolve residues 2 to 3 times by using ethyl acetate, combine all acetate layers and then wash the acetate layers twice by using purified water to remove water-soluble impurities; next, add active carbon to ethyl acetate phase for decolorization, stir for an hour, leach, wash, combine with acetate solution and then concentrate in vacuum till dried. After that, add 125 ml of water into residues, increase temperature to 65° C. to stir and dissolve, and a small amount of ethyl ether is then added in when temperature cools down below 40° C. By stirring, crystallization and filtration, raw sucralose-6-ester is obtained, ethyl acetate is re-crystallized and 20 g of refined sucralose-6-ester is obtained after drying (yield: 50%).

As mentioned before, the above description is intended only to describe the embodiments of the present invention. It is obvious that any person skilled in this field of art could partially change and modify the present invention without violating the present invention. The description above serves only as explanatory examples but not limitations of the present invention. Any method of preparing sucralose-6-ester by catalysis and chlorination of phase transfer catalyst containing the technical features of the present invention should fall within the scope of protection of the present invention.

What is claimed is:

1. A method of preparing sucralose-6-ester by catalysis and chlorination of phase transfer catalyst comprises the following steps: add phase transfer catalyst with sucrose-6-ester weighing 2-3% into a prepared Vilsmeier reagent and then drip in DMF solution of sucrose-6-ester with concentration of 15-25% in 5-10° C. to obtain a feed solution and after that maintain temperature of the feed solution for 1-1.5 hours; next, increase the temperature to 45-55° C. and maintain the temperature for 1-1.5 hours; next, increase the temperature to 75-85° C. and maintain the temperature for 1-1.5 hours; next, increase the temperature to 108-109° C., maintain the temperature for 2.5-3 hours and then remove a chlorine-containing solvent from the feed solution by concentration in reduced pressure for evaporation.

2. The method of preparing sucralose-6-ester by catalysis and chlorination of phase transfer catalyst as in claim 1, characterized in that, it also includes a step of cooling down the feed solution to room temperature and then pump in ammonia into the feed solution for neutralization.

3. The method of preparing sucralose-6-ester by catalysis and chlorination of phase transfer catalyst as in claim 2, characterized in that, pH value of the feed solution is adjusted to 6.5-7.5 after pumping in ammonia.

4. The method of preparing sucralose-6-ester by catalysis and chlorination of phase transfer catalyst as in claim 3, characterized in that, the phase transfer catalyst is one of the following: tetrabutyl ammonium bromide, tetrabutyl ammonium chloride, benzyl triethyl ammonium chloride, benzyl triethyl ammonium bromide, trioctylmethyl ammonium chloride, dodecyltrimethyl ammonium chloride or trimethyl-tetradecyl ammonium chloride.

5. The method of preparing sucralose-6-ester by catalysis and chlorination of phase transfer catalyst as in claim 4, characterized in that, the Vilsmeier reagent is prepared by first dissolving a chlorination reagent in a chlorine-containing solvent, namely, dichloroethane or trichloroethane, and then dripping resultant solution into a DMF solvent.

6. The method of preparing sucralose-6-ester by catalysis and chlorination of phase transfer catalyst as in claim 5, characterized in that, the chlorination reagent is one of the following: thionyl chloride, bis (trichloromethyl) carbonate or phosphorus oxychloride.
